(19) United States
(12) Patent Application Publication
(10) Pub. No.: US 2026/0298825 A1
(43) Pub. Date: Oct. 1, 2026

(54) METHODS AND SYSTEMS FOR DETERMINING NON-TRIAZOLE CORROSION INHIBITOR CONCENTRATION IN INDUSTRIAL WATER SYSTEMS

(71) Applicant: CHEMTREAT, INC., Glen Allen, VA (US)

(72) Inventor: Patrick WOOD, Midlothian, VA (US)

(73) Assignee: CHEMTREAT, INC., Glen Allen, VA (US)

(21) Appl. No.: 19/575,094

(22) Filed: Mar. 23, 2026

Related U.S. Application Data
(60) Provisional application No. 63/777,113, filed on Mar. 25, 2025.

(51) Int. Cl. G01N 21/64; C23F 11/14; G01N 33/18

(52) U.S. Cl. CPC G01N 21/643; C23F 11/145; G01N 33/1813; C23F 2213/10; G01N 2021/6439

(57) ABSTRACT

A method of determining an amount of a non-triazole corrosion inhibitor in water in a water system. The method includes adding a dye composition into a sample of the water. The dye composition reacts with the non-triazole corrosion inhibitor that is present in the sample of the water to form a complex of the dye composition and the non-triazole corrosion inhibitor. The method further includes adding an extraction solvent into the sample of water. The extraction solvent forms an organic layer that separates from an aqueous phase of the sample of water. Then, the method includes measuring absorbance of the organic layer at a wavelength in a range of about 440 to 495 nm; and determining the amount of the non-triazole corrosion inhibitor present in the water based on the measured absorbance.

### CROSS-REFERENCE TO RELATED APPLICATION

This application claims the benefit of U.S. Provisional Application No. 63/777,113 filed Mar. 25, 2025. The disclosure of the prior application is hereby incorporated by reference herein in its entirety.

### TECHNICAL FIELD

The present disclosure relates generally to methods and systems for determining the concentration of non-triazole corrosion inhibitors in industrial water systems.

### BACKGROUND

Corrosion of metal surfaces in water systems is a serious problem. Corrosion can cause undesirable consequences, including loss of heat transfer, increased cleaning frequency, equipment repairs and replacements, shutdowns, environmental problems and the increasing resources and costs associated with each. Some causes of increased corrosion of metal surfaces include high dissolved solids, acidic environments, elevated temperatures, microbiological growth, organic and mineral deposits, and fluids that contain relatively high concentration of gases such as oxygen, hydrogen sulfide, or carbon dioxide.

Copper and its alloys (all referred to generally as “yellow metals”) are commonly used in cooling water treatment systems for heat exchanger tubing, pump impellers, and various other applications due to the natural corrosion resistance and high thermal conductivity of these metals. However, copper and its alloys are not immune to corrosion in cooling water applications especially in the presence of halogen based oxidizing biocides such as hypochlorous acid (HOCl) or hypobromous acid (HOBr), which results in corrosion and possibly failure of heat exchangers.

Current corrosion inhibitors for copper and its alloys include triazole-based compounds, i.e., a heterocyclic compound that includes a five-membered ring of two carbon atoms and three nitrogen atoms. Conventional triazole corrosion inhibitors include tolyltriazole (TT), benzotriazole (BZT), and chlorinated tolyltriazole (Cl-TT). The triazoles work as yellow metal corrosion inhibitors by forming an inhibitor film on the surface of yellow metals through bonding with copper. However, the film formed by triazoles can be disrupted by halogen-based biocides (e.g., HOCl), which can lead to corrosion and equipment failure. The film formed by triazoles on the metal surface is also affected by high free chlorine and it requires additional triazole to re-passivate the film for corrosion protection. Additionally, in the bulk water, the triazole inhibitor can react and be degraded by halogen-containing biocide and its corrosion inhibition capacity reduced. Triazole inhibitors and their halogenated derivatives also have high aquatic toxicity which can limit their application in industrial cooling water treatment, and the raw materials required to manufacture triazoles are often impacted by cost fluctuation and supply chain vulnerability. The concentration of triazole inhibitors can be detected and monitored by the use of fluorescent agents, such as a reactive chemical tracer (e.g., PTSA).

Industrial water systems, including cooling water systems, present unique challenges in terms of efficacy, toxicity, and cost. Historically, phosphate and triazole-based inhibitor compositions have been considered. But these treatments lack stability and can be toxic. There is also a need for fast and accurate detection of non-triazole corrosion inhibitors in water systems so that the amount of the corrosion inhibitor present in the water system can be monitored and controlled, e.g., to avoid falling below a minimum threshold for inhibiting corrosion. These and other issues are addressed by the present disclosure.

### SUMMARY

Disclosed herein are non-triazole corrosion inhibitors that are effective to prevent corrosion of metal surfaces in contact with water, and a method of determining an amount of the non-triazole corrosion inhibitor in water in a water system. The non-triazole corrosion inhibitors include amide or amide derivatives. The method of determining an amount of the non-triazole corrosion inhibitor includes adding a dye composition into a sample of the water. The dye composition reacts with the non-triazole corrosion inhibitor that is present in the sample of the water to form a complex of the dye composition and the non-triazole corrosion inhibitor. The method further includes adding an extraction solvent into the sample of water. The extraction solvent forms an organic layer that separates from an aqueous phase of the sample of water. Then, the method includes measuring absorbance of the organic layer at a wavelength in a range of about 440 to 495 nm; and determining the amount of the non-triazole corrosion inhibitor present in the water based on the measured absorbance.

### BRIEF DESCRIPTION OF THE FIGURES

FIG. 1 is an example of a standard curve showing the relationship between the concentration of sodium cocoamphoacetate and absorbance at a wavelength of 450 nm.

### DETAILED DESCRIPTION OF EMBODIMENTS

In the following description, numerous details are set forth to provide an understanding of the present disclosure. However, it may be understood by those skilled in the art that the systems and methods of the present disclosure may be practiced without these details and that numerous variations or modifications from the described embodiments may be possible.

This disclosure relates to the use of non-triazole amide or amide derivatives that are effective to prevent corrosion of metal surfaces in contact with water, and methods for determining and monitoring the amount of the non-triazole corrosion inhibitors in the water. The non-triazole amide corrosion inhibitors overcome several of the drawbacks of known triazole-based corrosion inhibitors. In particular, disclosed non-triazole amide corrosion inhibitors have been shown to perform as better corrosion inhibitors for metal surfaces than conventional compounds used in cooling water corrosion inhibition applications. They have low halogen demand while exhibiting good corrosion resistance.

Disclosed non-triazole amide corrosion inhibitors form films on the surface of the metal and provide a hydrophobic barrier. This barrier prevents the transmission of corroding species like oxygen from interacting with the metal surface. These inhibitors and similar derivatives show excellent performance with yellow metals like copper and brass.

Methods for fast and accurate detection of the non-triazole corrosion inhibitors in a water system are also disclosed. The methods use colorimetric techniques for determining the concentration of the non-triazole corrosion inhibitors in the water in a water system, such as a cooling water system. The concentration can be monitored over time, enabling the concentration to be adjusted, for example, when a measured amount of corrosion inhibitor drops below a predetermined threshold.

### Corrosion Inhibitor Compounds

The non-triazole corrosion inhibitors can include compounds that are non-triazole derivatives of imidazoline and/or amide compounds having at least one carboxylic or carboxylate group and at least two nitrogen atoms. The number of the at least one carboxylic or carboxylate group can be in a range of, for example, 1 to 6, 1 to 4, or 2 to 4. The number of the at least two nitrogen atoms can be in a range of, for example, 2 to 6, 2 to 5, or 2 to 4. These compounds can include two nitrogens and at least 1 carboxylate group in the main chain. Preferably, the compounds include 1 to 2 carboxylate groups.

The non-triazole corrosion inhibitors are surfactants having an amphoteric structure. Amphoteric surfactants are uniquely structured to function as cationic surfactants at acid pH values and anionic surfactants at alkaline pH values. The non-triazole corrosion inhibotors can include amide compounds and amide derivatives having at least one carboxylic or carboxylate group and at least two nitrogen atoms. The number of the at least one carboxylic or carboxylate group can be in a range of, for example, 1 to 6, 1 to 4, or 2 to 4. The number of the at least two nitrogen atoms can be in a range of, for example, 2 to 6, 2 to 5, or 2 to 4. These compounds may include two nitrogens and at least 1 carboxylate group in the main chain.

The corrosion inhibitor can include alkylamphocarboxylates, which have been found to be particularly effective corrosion inhibitors on yellow metal and biodispersants. The alkylamphocarboxylates may have from 5 to 14 carbon atoms, or from 6 to 10 carbon atoms. For example, sodium cocoamphoacetate has been found to be an effective corrosion inhibitor and an effective biodispersant. The corrosion inhibitor can be any one or more of sodium cocoamphoacetate, disodium cocoamphodiacetate, disodium cocoamphodipropionate, disodium capryloamphodipropionate, disodium capryloamphodiacetate, and disodium lauroamphodiacetate. The corrosion inhibitor does not include any triazole groups, and preferably does not include any tetrazole groups.

The non-triazole compounds described above can be added to water that is in contact with a metal surface to inhibit or prevent corrosion of the metal surface. For example, the non-triazole compounds can be introduced into open or closed water systems. Further, the non-triazole compounds can be introduced into the water stream while the water system is on-line. The methods of inhibiting corrosion can be used in water systems including, but not limited to cooling water, cooling towers, water distribution systems, boilers, pasteurizers, water and brine carrying pipelines, storage tanks and the like. In general, water in these water systems is at least 90 wt. % water, at least 95 wt. % water, or at least 99 wt. % water.

The metal surface that is in contact with the treated water can include ferrous metals such as steel (e.g., mild steel, stainless steel, galvanized steel, etc.), aluminum and its alloys, and yellow metals (e.g., copper and copper-based alloys including bronzes, brasses, etc.). In one aspect, the disclosed non-triazole compounds are particularly useful in inhibiting corrosion of yellow metals. In this regard, the non-triazole compounds can prevent corrosion on yellow metals by forming an insoluble protective film on the surface. It is believed that the film is stabilized by a molecular bond with the organic inhibitor and copper and prevents surface interaction with corrosive species.

The water systems can be mixed metal systems. For example, mixed metal systems include systems that include surfaces of two or more metals such as copper or copper alloy metal surfaces and another metal. The other metals can include, but are not limited to, iron, silver, steel, zinc alloy, and aluminum. In embodiments, the other metal may be stainless steel, ferrous steel, and/or galvanized steel.

The water temperature in the water system may be from 0 to 200° C., from 0 to 180° C., from 0 to 140° C., from 1 to 120° C., from 1 to 100° C., from 20 to 70° C., from 40 to 60° C., or about 50° C. The water in the water system can be pressurized. The pH of the water may have a value of from 2 to 12, from 4 to 10, from 6 to 9, from 7.0 to 8.5, from 7.5 to 8.5, or about 8.

The non-triazole compounds described above can be introduced into the water in the water system in amounts that are effective to form a film of the non-triazole compound(s) on the metal surface and reduce corrosion of the metal surface to a desired degree. The non-triazole compounds can be added so that the non-triazole compounds are present in the water in amounts of from 0.1 ppm to 500 ppm, 0.5 ppm to 250 ppm, from 1 ppm to 100 ppm, 5 ppm to 50 ppm, or from 10 ppm to 25 ppm, for example. The non-triazole compounds can be added to the water continuously, periodically, or intermittently.

The non-triazole compounds can be added to the water in sufficient amounts and over a sufficient duration so that a protective film is formed on the corrodible metal surface, and in particular so that the protective film has a thickness that is in a range of from 1 to 150 nm, from 5 nm to 50 nm, or from 10 nm to 20 nm. The thickness of the protective film can be measured by applying a platinum coating to the corrodible metal surface (to enhance visibility of the protective film), imaging a cross-section of the corrodible metal surface with a Scanning Transmission Electron Microscopy (STEM), and then measuring the average protective film thickness from the STEM image as is known in the art. Forming a protective film of this thickness is evidence that inhibitor treatment has formed a uniform robust film that is effective to inhibit corrosion of the metal surface. In some embodiments, the film may take from 5 days to 120 days or from 15 days to 90 days with regular dosing of the inhibitor to achieve a suitable film thickness.

The non-triazole compounds can also increase the hydrophobicity of the corrodible metal surface, which reduces the wettability of the surface and thereby also reduces the corrosion potential. The increased hydrophobicity of the surface is also an indication that the non-triazole compounds have formed a protective film on the surface. The hydrophobicity of the corrodible metal surface can be quantified by placing a drop of water on the surface and measuring the contact angle of the droplet in accordance with ASTM D594. The treatment composition can be added in a sufficient amount and for a sufficient duration so that the treated metal surface exhibits a contact angle in a range of from 65 to 85 degrees, from 70 to 80 degrees, or from 72 to 78 degrees, for example. Similarly, the contact angle of the treated metal surface can increase by up to 40%, from 5% to 30%, or from 15% to 25%, for example, as compared to a like metal surface that is not treated with the treatment composition (i.e., having no protective film).

The compounds can be added to the water in response to a measured parameter of the water or of the metal surface, including when a measured amount of corrosion inhibitor drops below a predetermined threshold. The compounds can be added in response to a system demand of the system or surface demand of the metal surface.

### Methods for Determining and Monitoring an Amount of Corrosion Inhibitor in Water

Broadly speaking, the present disclosure further provides a method of determining an amount of a non-triazole corrosion inhibitor present in water in a water system, such as an industrial water system, for example, any of the water systems described above.

The method includes adding a dye composition and an extraction solvent into a sample of the water. The dye composition reacts with the non-triazole corrosion inhibitor that is present in the water to form a complex of the dye composition and the non-triazole corrosion inhibitor. The extraction solvent forms an organic layer that separates from an aqueous phase of the sample of water, and the complex of the dye composition and the non-triazole corrosion inhibitor moves into the organic layer. Then, the method includes measuring an absorbance of the organic layer at a particular wavelength; and determining the amount of the non-triazole corrosion inhibitor present in the water based on the measured absorbance. The absorbance has been found to be linear at typical treatment dosages. This provides a fast and reliable technique for determining the amount of non-triazole corrosion inhibitor in the water in a water system. This method can be used to monitor and control the amount of non-triazole corrosion inhibitor present in the water, and ensure that the concentration of non-triazole corrosion inhibitor does not fall below a predetermined threshold, for example, for satisfactorily inhibiting corrosion in the water system.

In the method, a sample of the water in the water system can be collected for analysis. The sample can be collected by a sample collector, such as a pipe or other vessel for collecting a sample from the water in the water system, or any other suitable device for collecting a sample of water. The sample can be collected automatically by a device or manually. For example, a portion of the water in the water system can be diverted into a pipe for vessel for analysis. The sample collector can be an open or closed system. A sample of any suitable amount can be collected. For example, the water sample can be collected in a range of 10 to 250 ml, 20 to 100 ml, or 30 to 50 ml.

Once the sample has been collected, a dye composition can be added to the sample. The dye composition can be any suitable dye that can react with the non-triazole corrosion inhibitor to form a complex. The complex between the non-triazole corrosion inhibitor and the dye composition can be soluble in the extraction solvent, which is discussed below. The dye composition can form a complex with the non-triazole corrosion inhibitor by ionic bonding, electrostatic interaction, and/or hydrogen bonding. For example, the dye can be an anionic dye, such as an azo dye. In an embodiment, the dye composition is Orange (II) dye. The dye composition can be added to the water sample in any suitable amount for forming a complex with the non-triazole corrosion inhibitor present in the water. For example, the dye composition can be added in an amount in a range of 0.1 to 20 wt. %, 0.5 to 10 wt. %, or 1 to 5 wt. % relative to the weight of the water sample.

An acid can be added to the water sample to reduce the pH of the water sample. For example, the acid can reduce the pH to a pH below 7, such as a pH within the range of 1 to 6.5, 1.5 to 6, or 2 to 5. At an acid pH (e.g., below 7), the non-triazole corrosion inhibitor that has an amphoteric structure (e.g., an amphoteric surfactant), which is present in the water, will act as a base to accept protons (H⁺), for example, from the acid. This will facilitate formation and extraction of a complex between the amphoteric surfactant and the anionic dye, such as Orange (II) to the organic layer, as discussed below. The acid can be added to the water sample before, after, or simultaneously with the dye composition. The acid can be, for example, sulfuric acid, hydrochloric acid, or phosphoric acid, or any other suitable acid for reducing the pH to facilitate formation and extraction of the complex. The acid can be added to the water sample in any suitable amount for reducing the pH to facilitate formation and extraction of the complex. For example, the acid can be added in an amount in a range of 0.1 to 20 wt. %, 0.5 to 10 wt. %, or 1 to 5 wt. % relative to the weight of the water sample.

An extraction solvent can be introduced into the water sample before, after, or simultaneously with the dye composition and/or the acid. In an embodiment, the extraction solvent is introduced in the water sample after the dye composition. The extraction solvent can be any suitable solvent that, when introduced into the water sample, can form an organic layer that separates from the aqueous phase of the water sample. For example, the extraction solvent can be chloroform or dichloromethane, or any other suitable extraction solvent. The extraction solvent can be added to the water sample in any suitable amount for forming the organic layer. For example, the extraction solvent can be added into the water sample in an amount in a range of 5 wt. % to 100 wt. %, 10 wt. % to 50 wt. %, or 15 wt. % to 25 wt. % relative to the weight of the water sample.

After the extraction solvent is introduced into the water sample, the sample vessel can be shaken (e.g., vigorously shaken) and/or centrifuged to facilitate separation of the two layers (i.e., the aqueous and organic layers). For example, the sample vessel can be shaken for any suitable timeframe for facilitating separation of the aqueous and organic layers, such as an amount of time in a range of 10 seconds to 15 minutes, 30 seconds to 10 minutes, 1 to 7 minutes, or 2 to 5 minutes. Alternatively or additionally, the sample vessel can be subjected centrifugation to separate the phases, for example, if the sample contains higher concentration of the non-triazole corrosion inhibitor. After shaking and/or centrifugation, the sample vessel can be allowed to stand for an amount of time, such as 1 to 15 minutes, 2 to 10 minutes, or 3 to 5 minutes, to allow the aqueous and organic phases to be completely separated. The organic layer will sink below the aqueous layer due to the higher density of the extraction solvent, such as chloroform, than water. Without being bound by theory, it is believed that the complex of the dye composition and the non-triazole corrosion inhibitor moves into the organic layer formed by the extraction solvent due to its solubility in the extraction solvent.

Once the organic and aqueous layers are separated, the absorbance of the organic layer can be measured in order to determine the amount of the non-triazole corrosion inhibitor in the water. Due to the presence of the non-triazole corrosion inhibitor in the organic layer, the absorbance differs from the absorbance of the extraction solvent (i.e., without the complex of the dye and the non-triazole corrosion inhibitor). For example, the absorbance can be increased due to the presence of the non-triazole corrosion inhibitor in the organic layer as compared to the absorbance of the extraction solvent alone. The change (e.g., increase) in absorbance can be used to determine the amount of the non-triazole corrosion inhibitor present in the water in the water system.

To measure the absorbance of the organic layer, one or more aliquots of the organic layer can be pipetted from the bottom organic layer present in the sample vessel. For example, one or more aliquots of the organic layer can be pipetted and discarded to condition the pipette. Then, a further aliquot (e.g., a second, third, fourth, etc. aliquot) of the organic layer can be pipetted and placed in a cuvette or other vessel for measuring the absorbance. Preferably, the cuvette or other vessel for measuring the absorbance of the organic layer contains no water, which could cause erroneous absorbance readings. The absorbance can be measured at a wavelength in a range of about 440 to 495 nm, such as 450 nm. The absorbance can be measured using any suitable device, such as a spectrophotometer. A spectrophotometer uses a light source that emits light at a specific wavelength or across a range of wavelengths so that the light passes through the sample being analyzed (i.e., the organic layer or the aliquot thereof) and measures the amount of light that is absorbed by the sample. The absorbance can be measured against a blank of the extraction solvent (e.g., a chloroform blank) to determine the change (e.g., increase) in absorbance. This change (e.g., increase) in the absorbance can be attributed to the presence of the complex of the non-triazole corrosion inhibitor and the dye composition in the organic layer, and can be correlated to the amount of the non-triazole corrosion inhibitor present in the water.

A standard curve can be determined from the relationship between the change (e.g., increase) in absorbance of the organic layer and the concentration of the non-triazole corrosion inhibitor so that the amount of the non-triazole corrosion inhibitor in the water system can be quantified. For example, to determine the standard curve, water samples containing various known concentrations of the non-triazole corrosion inhibitor can be prepared and analyzed according to the method described herein. The increase in absorbance of the organic layers formed in the samples containing the known concentrations of the non-triazole corrosion inhibitor can be measured. The increase absorbance can then be plotted against the known concentrations of the non-triazole corrosion inhibitor, and a regression of these data points can be performed.

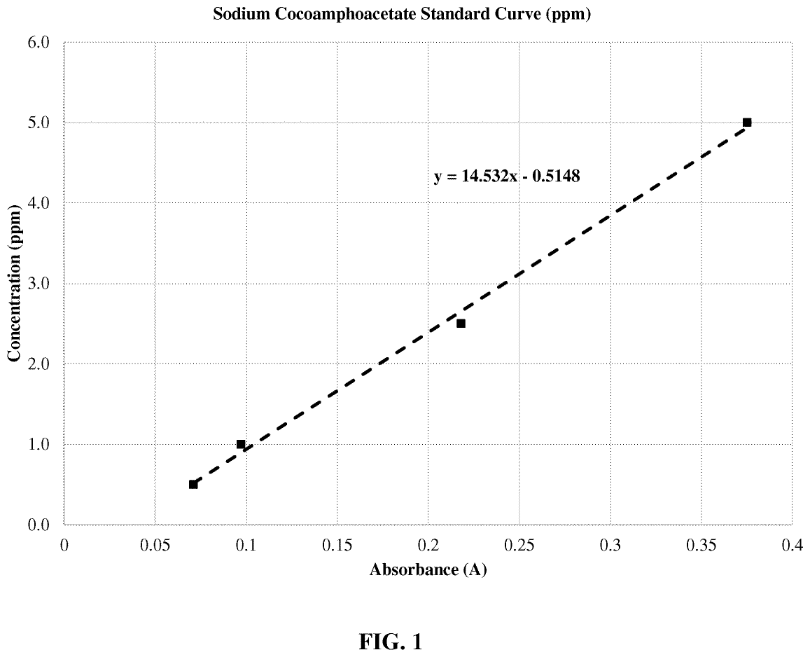

For example, FIG. 1 shows an example of a standard curve for sodium cocoamphoacetate. FIG. 1 shows a substantially linear relationship (with an r² value of about 0.997) between the absorbance and the concentration of sodium cocoamphoacetate. As discussed in more detail in Example 1, known amounts of sodium cocoamphoacetate were added to water samples collected in a vessel. Sulfuric acid, Orange (II) dye, and chloroform were added to the water samples, and an organic layer was formed and separated from the aqueous phase of the samples. The absorbance of the organic layer in each sample was then measured at a wavelength of 450 nm against a chloroform blank to determine the increase in absorbance due to the presence of the sodium cocoamphoacetate in the known amounts. In FIG. 1, the y-axis numbers represent the known amounts of sodium cocoamphoacetate in the samples. The measured increase in the absorbance was then plotted against the known concentrations of sodium cocoamphoacetate in the samples, and a regression of these data points was performed to produce the standard curve shown in FIG. 1. The graph in FIG. 1 shows that the increase in absorbance is substantially proportional to the amount of sodium cocoamphoacetate (on the y-axis) present in the samples.

Once a standard curve has been generated for a non-triazole corrosion inhibitor, it can be used to determine the amount of the non-triazole corrosion inhibitor in the water in the water system. For example, a sample of water from the water system can be collected and analyzed according to the method described herein. The measured increase in absorbance of the organic layer (containing the complex of the dye composition and the non-triazole corrosion inhibitor) can be compared to the standard curve to determine the corresponding concentration of the non-triazole corrosion inhibitor.

The methods described herein enable the target non-triazole corrosion inhibitor to be accurately quantified by a quick and simple technique. The disclosed methods can provide valuable real-time information regarding corrosion inhibitor levels in water systems, which in turn can be used to control the amount of corrosion inhibitor that is present in and added to the system, for example, to ensure that the amount of the non-triazole corrosion inhibitor that is present in the water in the water system does not fall below a minimum threshold for inhibiting corrosion.

The present disclosure further relates to a method for monitoring and controlling the amount of a non-triazole corrosion inhibitor in the water in a water system. The amount of the non-triazole corrosion inhibitor can be continuously, periodically, or intermittently monitored and controlled. For example, the method can involve repeatedly (e.g., periodically, intermittently, or continuously) determining the amount of the corrosion inhibitor in the water according to the methods described herein (e.g., by collecting a water sample, introducing a dye composition and extraction solvent into the water sample, and measuring the absorbance of the organic layer that is formed in the water sample to determine the amount of the corrosion inhibitor, for example, from a standard curve) to monitor the concentration of the non-triazole corrosion inhibitor in the water system.

The method can further include adjusting the amount of the non-triazole corrosion inhibitor in the water based on the determined amount. For example, the amount of the non-triazole corrosion inhibitor can be adjusted to be within a predetermined range, for example, for sufficiently inhibiting corrosion in the water system. The predetermined range can be a range for achieving sufficient corrosion protection. For example, if the determined amount of the corrosion inhibitor in the water is below the predetermined range, more of the corrosion inhibitor concentration can be added into the water to increase the amount of corrosion inhibitor in the water in the water system to a value within the predetermined range, and if the determined corrosion inhibitor concentration is greater than the predetermined range, extraction, dilution, or neutralization methods can be performed to decrease the concentration of corrosion inhibitor in the water to a value within the predetermined range. As a non-limiting example, predetermined range can be a range of from 0.1 ppm to 500 ppm, 0.5 ppm to 250 ppm, from 1 ppm to 100 ppm, 5 ppm to 50 ppm, or from 10 ppm to 25 ppm.

The amount of the non-triazole corrosion inhibitor present in the water in the water system can be adjusted by comparing the determined amount to one or more predetermined threshold values. For example, the determined corrosion inhibitor concentration can be compared to a first threshold value, which can be a minimum corrosion inhibitor concentration for obtaining adequate corrosion protection or a desired level of corrosion inhibition, a value just above the minimum corrosion inhibitor concentration for obtaining adequate or desirous corrosion protection, or any other suitable value. If the determined corrosion inhibitor concentration is below the threshold value, then more corrosion inhibitor can be introduced into the water in the water system to increase the corrosion inhibitor concentration to a value greater than the threshold value.

The determined corrosion inhibitor concentration can also or alternatively be compared to a second threshold value, which can be a value of a desired maximum corrosion inhibitor concentration in the water or any other suitable value. For example, the maximum amount can be an amount beyond which the addition of more of the corrosion inhibitor only produces negligible increases in corrosion inhibition or an amount beyond which the addition or more corrosion inhibitor is considered wasteful. If the determined concentration is greater than the second threshold, then extraction, neutralization, and/or dilution methods can be performed to decrease the corrosion inhibitor concentration in the water to a value below the second threshold. Accordingly, the concentration of the corrosion inhibitor in the water can be adjusted by adding more of the corrosion inhibitor into the water if the determined amount is below a first threshold, or by decreasing the concentration of the corrosion inhibitor in the water if the determined amount is equal to or greater than a second threshold. Continuous, periodic, or intermittent monitoring enables the concentration of the non-triazole corrosion inhibitor in the water to be controlled to be within a desired predetermined range.

The method can include repeatedly determining the amount of the non-triazole corrosion inhibitor in the water according to the methods disclosed herein over a period of time. For example, the amount of the corrosion inhibitor can be determined at predetermined intervals, such as every hour, every 2 hours, daily, or weekly, or any other suitable interval. Then, the amount of the corrosion inhibitor in the water can be adjusted depending on the determined amount.

The amount of the corrosion inhibitor in the water can be adjusted automatically or manually. For example, a controller or processor can be configured to control a corrosion inhibitor delivery device to introduce more of the corrosion inhibitor into the water in the water system. The corrosion inhibitor delivery device can be a pump or any other suitable device for pumping or otherwise delivering the corrosion inhibitor from a storage tank, reservoir, or other storage container storing the corrosion inhibitor into the water in the water system. The controller can control the delivery device to introduce a specific amount of the corrosion inhibitor into the water so that the amount of the corrosion inhibitor in the water is within a predetermined range or above a predetermined threshold, as discussed above.

A processor or controller, which can be the same or different from the controller for controlling the delivery device, can be configured to determine the amount of the corrosion inhibitor from the measured absorbance. For example, the controller can receive the absorbance measured by the spectrophotometer, and can compare the measured increase in the absorbance to the standard curve to determine the amount of the non-triazole corrosion inhibitor present in the water. Based on the calculated amount of the corrosion inhibitor present in the water, the controller can send instructions for modifying the process conditions, for example, by controlling the corrosion inhibitor delivery device to introduce more of the corrosion inhibitor (e.g., a specific amount thereof) into the water in the water system.

The controller(s) and processor(s) described above can each independently be a processor, microprocessor, CPU, or any other suitable device for receiving, processing, analyzing, and recording information, including absorbance results from the spectrophotometer and any other device, and transmitting instructions and/or command signals to other devices based on the received information. Data can also be stored in a memory. For example, the standard curve and threshold amounts or predetermined ranges of the corrosion inhibitor can be stored in the memory. The memory can be in the form of any computer data storage, such as random-access memory or flash memory.

Although some embodiments have been described in detail above, those skilled in the art will readily appreciate that many modifications are possible in the example embodiments without materially departing from the disclosed embodiments. Accordingly, all such modifications are intended to be included within the scope of this disclosure. The foregoing is further illustrated by reference to the following example, which is presented for purposes of illustration and is not intended to limit the scope of the present disclosure.

### EXAMPLES

A standard curve that correlates measured absorbance to concentration levels of sodium cocoamphoacetate was generated according to the following Example.

### Reagents

Acid Reagent: An 18N solution of sulfuric acid was prepared by slowly and gently adding concentrated sulfuric acid (100 ml) to distilled or deionized water (100 ml).

Dye Reagent: 1 g of Orange (II) (Sigma Ltd, 95% dye content) was dissolved in 1 liter of deionized or distilled water containing 0.1 M sodium chloride (5.9 g).

Extraction Solvent Reagent: Analytical reagent grade chloroform.

### Example

Four water samples (40 ml) containing 0.5 ppm, 1.0 ppm, 2.5 ppm, and 5.0 ppm of sodium cocoamphoacetate were prepared and each sample was placed in a suitable extraction vessel. Then, 0.8 ml of the sulfuric acid solution, 0.8 ml of the Orange(II) reagent, and 7.5 ml of chloroform was added to each of the four water samples. Each extraction vessel was shaken vigorously for 2 minutes, and the two layers left to separate.

For each sample, a clean Pasteur pipette was used to pipette off an aliquot of the bottom organic layer containing the chloroform extract, which was discarded, in order to condition the pipette. This was repeated, and then the third aliquot was placed in a clean 1 cm cuvette. The absorbance was measured by a spectrophotometer at a wavelength of 450 nm against a chloroform blank. The measured absorbance values (i.e., the increase in absorbance compared to the chloroform blank) were recorded and plotted against the known concentrations of sodium cocoamphoacetate in the four samples, and a regression of these data points was performed to produce the standard curve shown in FIG. 1.

The graph in FIG. 1 shows that the measured absorbance (on the x-axis) is substantially proportional to the amount of sodium cocoamphoacetate (on the y-axis) present in the samples. In particular, FIG. 1 shows a substantially linear relationship with an r² value of about 0.997 between the absorbance and the sodium cocoamphoacetate concentration. As shown in FIG. 1, the relationship between the concentration of sodium cocoamphoacetate and the measured absorbance can be described according to the below equation:

$$Concentration of Sodium Cocoamphoacetate = 14.532 * ( Absorbance ) - 0.5148$$

The standard curve shown in FIG. 1 can be used to determine the concentration of sodium cocoamphoacetate in water containing unknown amounts of sodium cocoamphoacetate by performing the methods described herein, and comparing the measured absorbance to the standard curve to determine the amount of sodium cocoamphoacetate.

It will be appreciated that the above-disclosed embodiments, features, and functions, or alternatives thereof, may be desirably combined into different methods or systems. Also, various alternatives, modifications, variations or improvements may be subsequently made by those skilled in the art, and are also intended to be encompassed by the following claims. As such, various changes may be made without departing from the spirit and scope of this disclosure as defined in the claims.

What is claimed is:

1. A method of determining an amount of a non-triazole corrosion inhibitor in water in a water system, the method comprising:
adding a dye composition into a sample of the water, wherein the dye composition reacts with the non-triazole corrosion inhibitor that is present in the sample of the water to form a complex of the dye composition and the non-triazole corrosion inhibitor;
adding an extraction solvent into the sample of water, wherein the extraction solvent forms an organic layer that separates from an aqueous phase of the sample of water;
measuring absorbance of the organic layer at a wavelength in a range of about 440 to 495 nm; and
determining the amount of the non-triazole corrosion inhibitor present in the water based on the measured absorbance.

2. The method according to claim 1, wherein the non-triazole corrosion inhibitor is a surfactant having an amphoteric structure.

3. The method according to claim 1, wherein the non-triazole corrosion inhibitor is an amide compound or amide derivative.

4. The method according to claim 1, wherein the non-triazole corrosion inhibitor is selected from the group consisting of sodium cocoamphoacetate, disodium cocoamphodiacetate, disodium cocoamphodipropionate, disodium capryloamphodipropionate, disodium capryloamphodiacetate, and disodium lauroamphodiacetate.

5. The method according to claim 1, wherein the water in the water system contacts a yellow metal surface, and the non-triazole corrosion inhibitor forms a film on the yellow metal surface to inhibit corrosion of the yellow metal surface.

6. The method according to claim 1, wherein the water system is a cooling water system, cooling tower, water distribution system, boiler, pasteurizer, water and brine carrying pipeline, or a storage tank.

7. The method according to claim 1, further comprising collecting the sample of the water from the water system.

8. The method according to claim 1, further comprising adding an acid into the sample of water to reduce the pH of the sample to below 7.

9. The method according to claim 8, wherein the pH is reduced to within a range of 1 to 6.

10. The method according to claim 8, wherein the acid is sulfuric acid, hydrochloric acid, or phosphoric acid.

11. The method according to claim 1, wherein the dye composition is Orange (II) dye.

12. The method according to claim 1, wherein the extraction solvent is chloroform or dichloromethane.

13. The method according to claim 1, wherein the amount of the non-triazole corrosion inhibitor present in the water is determined by comparing the measured absorbance with a standard curve that correlates absorbance with concentrations of the non-triazole corrosion inhibitor.

14. The method according to claim 1, wherein after the organic layer is formed in the sample of water, the complex of the dye composition and the non-triazole corrosion inhibitor moves into the organic layer.

15. The method according to claim 14, wherein:
the absorbance of the organic layer is measured against a blank of the extraction solvent to determine the change in absorbance between the extraction solvent and the organic layer; and
the amount of the non-triazole corrosion inhibitor present in the water is determined by comparing the measured change in absorbance with a standard curve that correlates the change in absorbance with concentrations of the non-triazole corrosion inhibitor.

16. A method of monitoring and controlling an amount of a non-triazole corrosion inhibitor in water in a water system, the method comprising:
performing the method according to claim 1 to determine the amount of the non-triazole corrosion inhibitor in the water; and
adjusting the amount of the non-triazole corrosion inhibitor in the water based on the determined amount of the non-triazole corrosion inhibitor.

17. The method according to claim 16, wherein the amount of the non-triazole corrosion inhibitor is adjusted to be within a predetermined range.

18. The method according to claim 17, wherein the amount of the non-triazole corrosion inhibitor is adjusted to be within a range of 0.1 ppm to 100 ppm.

19. The method according to claim 16, wherein the amount of the non-triazole corrosion inhibitor is repeatedly determined over a period of time, and the amount is adjusted when the determined amount is below a predetermined threshold amount.
